SEQUENCE LISTING (2 sequences)

SEQ ID NO: 1 - protein, 52 aa, Homo sapiens
YRQSMNNFQGLRSFGCRFGTCTVQKLAHQIYQFTDKDKDNVAPRSKISPQGY

SEQ ID NO: 2 - protein, 461 aa, Homo sapiens
MEKKCTLNFLVLLPFFMILVTAELEESPEDSIQLGVTRNKIMTAQYECYQKIMQDPIQQAEGVYCNRTWDGWLCWNDVAAGTESMQLCPDYFQDFDPSEKVTKICDQDGNWFRHPASNRTWTNYTQCNVNTHEKVKTALNLFYLTIIGHGLSIASLLISLGIFFYFKSLSCQRITLHKNLFFSFVCNSVVTIIHLTAVANNQALVATNPVSCKVSQFIHLYLMGCNYFWMLCEGIYLHTLIVVAVFAEKQHLMWYYFLGWGFPLIPACIHAIARSLYYNDNCWISSDTHLLYIIHGPICAALLVNLFFLLNIVRVLITKLKVTHQAESNLYMKAVRATLILVPLLGIEFVLIPWRPEGKIAEEVYDYIMHILMHFQGLLVSTIFCFFNGEVQAILRRNWNQYKIQFGNSFSNSEALRSASYTVSTISDGPGYSHDCPSEHLNGKSIHDIENVLLKPENLYN

(19) United States
(12) Patent Application Publication
(10) Pub. No.: US 2023/0037414 A1
(43) Pub. Date: Feb. 9, 2023

(54) INHIBITORS OF ADRENOMEDULLIN FOR THE TREATMENT OF ACUTE MYELOID LEUKEMIA BY ERADICATING LEUKEMIC STEM CELLS

(71) Applicants: INSERM (INSTITUT NATIONAL DE LA SANTÉ ET DE LA RECHERCHE MÉDICALE, Paris (FR); UNIVERSITÉ TOULOUSE III - PAUL SABATIER, Toulouse (FR)

(72) Inventors: Jean-Emmanuel SARRY, Toulouse (FR); Clément LARRUE, Toulouse (FR)

(21) Appl. No.: 17/778,446

(22) PCT Filed: Nov. 20, 2020
(86) PCT No.: PCT/EP2020/082955

§ 371 Date: May 20, 2022

(30) Foreign Application Priority Data
Nov. 22, 2019 (EP) 19306513.3

(51) Int. Cl. C07K 16/28; A61K 31/713; A61P 35/02

(52) U.S. Cl. CPC C07K 16/2869; A61K 31/713; A61P 35/02

(57) ABSTRACT

The emergence of cells with drug resistant and/or stem cell features might explain frequent relapses and the poor outcome of patients with acute myeloid leukemia (AML). LSCs are heterogeneous for their phenotypes and their sensitivity to chemotherapeutic agents in vivo. Using in silico and functional approaches, the inventors uncovered that CALCRL is overexpressed in LSCs compared with normal hematopoietic cells. They further demonstrated that the CALCRL ligand adrenomedullin (ADM) is highly expressed in AML cells and that increased transcript level was markedly associated with decreased complete remission rates, 5-year overall and event7free survival. The inventors also showed that CALCRL depletion strongly affected leukemic growth in vivo and increased mice survival. Targeting ADM phenocopies the biological and anti-leukemic effects of the CALCRL depletion. These data highlight the critical role of ADM and disclose a promising therapeutic target to specifically eradicate R-LSCs and overcome relapse in AML.

### FIELD OF THE INVENTION

The present invention is in the field of medicine, in particular oncology.

### BACKGROUND OF THE INVENTION

Acute myeloid leukemia (AML) arises from self-renewing leukemic stem cells (LSCs) which can repopulate human AML when assayed and xenografted in immunocompromised mice (e.g. PDX; Bonnet and Dick, 1997). Despite the fact that these cells represent a tiny minority of all leukemic cells, the fact that gene signatures associated with a stem cell phenotype or function are associated with an unfavorable prognosis in AML, strongly supports the hypothesis that their abundance has a real clinical impact (Gentles et al., 2010; Vergez et al., 2011; Eppert et al., 2011; Ng et al., 2016). This clinical relevance is supported by studies showing that relapsing patients presents a constantly enrichment of LSC frequency (Ho et al., 2016) and an increase of LSC-related gene signatures (Hackl et al., 2015). While it has been initially shown that LSCs are spared from chemotherapy (Jordan et al., 2006; Ishikawa et al., 2007), recent works demonstrated that cytarabine (AraC) can have a strong impact on the LSC pool in PDX models and patients (Farge et al., 2017; Boyd et al., 2018). These results suggest that there are two distinct LSC populations, some chemosensitive (S-LSCs) and thus eradicated by conventional treatments, and others that are chemoresistant (R-LSCs) persist, regenerate AML and initiate the relapse of patients. Thus, better phenotypically and functionally characterizing these R-LSCs is crucial to allow the development of new therapy strategies that specifically target R-LSCs.

Although it has been first proposed that the LSC-compartment was restricted to the CD34⁺CD38⁻ subpopulation of human AML cells (Bonnet and Dick, 1997; Ishikawa et al., 2007), several studies subsequently demonstrated that LSCs are also phenotypically heterogeneous such as for instance CD38⁺ AML cells or CD34⁻ cells from NPM1c-mutated specimens are also able to serially recapitulate the disease when assayed in NSG-deficient mice (Taussig et al., 2008; Taussig et al., 2010; Sarry et al., 2011; Quek et al., 2016). This highlights that more functional studies are needed to well characterize LSCs (Eppert et al., 2011).

Eradicating R-LSCs without killing normal hematopoietic stem cells (HSCs) depends on identifying markers overexpressed in AML compartment and functionally relevant. In recent years, many research efforts to distinguish LSCs from HSCs have been made and allowed the identification of several cell surface markers such as CD47, CD123, CD44, TIM-3, CD25, CD32 or CD93 (Majeti et al., 2009; Jin et al., 2009; Kikushige et al., 2010; Saito et al., 2010; Iwasaki et al., 2015). In addition to these new membrane markers, it has been proposed that LSCs have also a specific increase in BCL2-dependent oxidative phosphorylation (OxPHOS), revealing a Achille's heel (vulnerability) that could be exploited through the treatment with BCL2 inhibitors such as ABT-199 (Lagadinou et al., 2013; Konopleva et al., 2016). This is consistent with several studies including our recent one that demonstrate that mitochondrial OxPHOS status contributes to drug resistance in leukemia (Farge et al., 2017; Bosc et al. 2017; Kunst et al. 2017). Taken together, all these results suggest specific characteristics of R-LSCs that leave an open door to targeted therapeutic approaches aimed at eradicating these cells.

### SUMMARY OF THE INVENTION

As defined by the claims, the present invention relates to inhibitors of adrenomedullin for the treatment of acute myeloid leukemia by eradicating leukemic stem cells.

### DETAILED DESCRIPTION OF THE INVENTION

After intensive chemotherapy, the emergence of cells with drug resistant and/or stem cell features might explain frequent relapses and the poor outcome of patients with acute myeloid leukemia (AML). Previous reports have shown that LSCs are heterogeneous for their phenotypes and their sensitivity to chemotherapeutic agents in vivo. This indicates that new drugs should selectively target drug-resistant/residual leukemic stem cell (hereafter R-LSC) subpopulations responsible for relapse. Using in silico and functional approaches, the inventors uncovered that the 7TMD G-protein coupled receptor CALCRL is preferentially overexpressed in LSCs compared with normal hematopoietic cells. They further demonstrated that the CALCRL ligand adrenomedullin (ADM) is highly expressed in AML cells and that increased transcript levels of CALCRL and ADM were markedly associated with decreased complete remission rates, 5-year overall and event-free survival in AML. Furthermore, CALCRL knock-down decreased LSC frequency and CALCRL^{high} (but not CALCRL^{low}) sorted cell subpopulations efficiently engrafted in mice. Accordingly, we showed that CALCRL depletion strongly affected leukemic growth in vivo and increased mice survival. Interestingly, CALCRL expression predicted the response of ten PDX models to cytarabine in vivo and its silencing sensitized cells to this drug in vivo. Targeting ADM phenocopies the biological and anti-leukemic effects of the CALCRL depletion. Mechanistically, the inventors showed that ADM-CALCRL axis drove cell cycle, DNA integrity and high mitochondrial OxPHOS function of AML blasts in an E2F1- and BCL2-dependent manner, all consistent with a drug tolerant status. Finally, the inventors demonstrated that CALCRL-depletion in resistant/residual AML cells after in vivo treatment with cytarabine impaired leukemic engraftment and LSC frequency when assayed in secondary transplant. All of these data highlight the critical role of CALCRL and ADM in residual stem cell survival, proliferation and metabolism and disclose a promising therapeutic target to specifically eradicate R-LSCs and overcome relapse in AML.

The first object of the present invention relates to a method of depleting leukemic stem cells in a subject suffering from AML comprising administering to the subject a therapeutically effective amount of an inhibitor of adrenomedullin activity or expression thereby depleting said leukemic stem cells.

As used herein, the term “acute myeloid leukemia” or “acute myelogenous leukemia” (“AML”) refers to a cancer of the myeloid line of blood cells, characterized by the rapid growth of abnormal white blood cells that accumulate in the bone marrow and interfere with the production of normal blood cells.

As used herein, the term “leukemic stem cell” has its general meaning in the art and refers to a pluripotent myeloid stem cell characterized by genetic transformation resulting in unregulated cell division. The leukaemic stem cells (LSC) are distinguished from all other AML cells by self-renewal ability, i.e. the ability to generate daughter cells similar to the mother one. The extensive self-renewal ability is an intrinsic property of LSC, and has been shown essential for the development of leukaemia.

As used herein, the term “deplete” with respect to leukemic stem cells, refers to a measurable decrease in the number of leukemic stem cells in the subject. The reduction can be at least about 10%, e.g., at least about 20%, 30%, 40%, 50%, 60%, 70%, 80%, 90%, 95%, 96%, 97%, 98%, 99%, or more. In some embodiments, the term refers to a decrease in the number of leukemic stem cells in a subject or in a sample to an amount below detectable limits.

The method of the present invention is thus particularly suitable for the treatment of AML.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

A further object of the present invention relates to a method for preventing relapse of a patient suffering from AML who was treated with chemotherapy comprising administering to the subject a therapeutically effective amount of an inhibitor of adrenomedullin activity or expression.

As used herein, the term “relapse” refers to the return of cancer after a period of improvement in which no cancer could be detected. Thus, the method of the present invention is particularly useful to prevent relapse after putatively successful treatment with chemotherapy.

Accordingly, a further object of the present invention relates to a method of treating chemoresistant acute myeloid leukemia (AML) in a patient in need thereof comprising administering to the patient a therapeutically effective amount of an inhibitor of adrenomedullin activity or expression.

As used herein; the term “chemoresistant acute myeloid leukemia” refers to the clinical situation in a patient suffering from acute myeloid leukemia when the proliferation of cancer cells cannot be prevented or inhibited by means of a chemotherapeutic agent or a combination of chemotherapeutic agents usually used to treat AML, at an acceptable dose to the patient. The leukemia can be intrinsically resistant prior to chemotherapy, or resistance may be acquired during treatment of leukemia that is initially sensitive to chemotherapy.

As used herein, the term “chemotherapeutic agent” refers to any chemical agent with therapeutic usefulness in the treatment of cancer. Chemotherapeutic agents as used herein encompass both chemical and biological agents. These agents function to inhibit a cellular activity upon which the cancer cell depends for continued survival. Categories of chemotherapeutic agents include alkylating/alkaloid agents, antimetabolites, hormones or hormone analogs, and miscellaneous antineoplastic drugs. Most if not all of these drugs are directly toxic to cancer cells and do not require immune stimulation. Suitable chemotherapeutic agents are described, for example, in Slapak and Kufe, Principles of Cancer Therapy, Chapter 86 in Harrison's Principles of Internal medicine, 14th edition; Perry et at, Chemotherapeutic, Ch 17 in Abeloff, Clinical Oncology 2nd ed., 2000 ChrchillLivingstone, Inc.; Baltzer L. and Berkery R. (eds): Oncology Pocket Guide to Chemotherapeutic, 2nd ed. St. Louis, mosby-Year Book, 1995; Fischer D. S., Knobf M. F., Durivage H J. (eds): The Cancer Chemotherapeutic Handbook, 4th ed. St. Louis, Mosby-Year Handbook.

In some embodiments the chemotherapeutic agent is cytarabine (cytosine arabinoside, Ara-C, Cytosar-U), quizartinib (AC220), sorafenib (BAY 43-9006), lestaurtinib (CEP-701), midostaurin (PKC412), carboplatin, carmustine, chlorambucil, dacarbazine, ifosfamide, lomustine, mechlorethamine, procarbazine, pentostatin, (2′deoxycoformycin), etoposide, teniposide, topotecan, vinblastine, vincristine, paclitaxel, dexamethasone, methylprednisolone, prednisone, all-trans retinoic acid, arsenic trioxide, interferon-alpha, rituximab (Rituxan®), gemtuzumab ozogamicin, imatinib mesylate, Cytosar-U), melphalan, busulfan (Myleran®), thiotepa, bleomycin, platinum (cisplatin), cyclophosphamide, Cytoxan®), daunorubicin, doxorubicin, idarubicin, mitoxantrone, 5-azacytidine, cladribine, fludarabine, hydroxyurea, 6-mercaptopurine, methotrexate, 6-thioguanine, or any combination thereof. In some embodiments, the leukemia is resistant to a combination of daunorubicin plus cytarabine (AraC), or idarubicin plus cytarabine (AraC).

In some embodiments, the chemotherapeutic agent is a BCL2 inhibitor. In some embodiments, the Bcl-2 inhibitor comprises 4-(4-{[2-(4-chlorophenyl)-4,4-dimethylcyclohex-1-en-1-yl]methyl}piperazin-1-yl)-N-({3-nitro-4-[(tetrahydro-2H-pyran-4-ylmethyl)amino]phenyl}sulfonyl)-2-(1H-pyrrolo[2,3-b]pyridin-5-yloxy)benzamide (also known as, and optionally referred to herein as, venetoclax, or ABT-199, or GDC-0199) or a pharmaceutically acceptable salt thereof.

In some embodiments, the chemotherapeutic agent is a FLT3 inhibitor. Examples of FLT3 inhibitors include N-(2-diethylaminoethyl)-5-[(Z)-(5-fluoro-2-oxo-1 H-indol-3-ylidene)methyl]-2,4-dimethyl-1 H-pyrrole-3-carboxamide, sunitinib, also know as SU11248, and marketed as SUTENT (sunitinib malate); 4-[4-[[4-chloro-3-(trifluoromethyl)phenyl]carbamoylamino]phenoxy]-N-methyl-pyridine-2-carboxamide, sorafenib, also known as BAY 43-9006, marketed as NEXAVAR (sorafenib); (9S,10R,1 1R,13R)-2,3, 10,11, 12,13-Hexahydro-10-methoxy-9-methyl-1 1-(methylamino)-9,13-epoxy-1H,9H-diindolo[1,2,3-gh: 3′,2′,1′-1m]pyrrolo[3,4-j][1,7]benzodiamzonine-1-one, also know as midostaurin or PKC412; (5S,6S,8R)-6-Hydroxy-6-(hydroxymethyl)-5-methyl-7,8,14,15-tetrahydro-5H-16-oxa-4b,8a,14-triaza-5,8-methanodibenzo[b,h]cycloocta[jkl]cyclopenta[e]-as-indacen-13(6H)-one, also know as lestaurtinib or CEP-701; 1-(5-(tert-Butyl)isoxazol-3-yl)-3-(4-(7-(2-morpholinoethoxy)benzo[d]imidazo[2,1-b]thiazol-2-yl)phenyl)urea, also known as Quizartinib or AC220; 1-(2-{5-[(3-Methyloxetan-3-yl)methoxy]-1H-benzimidazol-1-yl}quinolin-8-yl)piperidin-4-amine, also known as Crenolanib or CP-868,596-26. See, e.g., Wander S. A., TherAdv Hematol. 5: 65-77 (2014). Other FLT3 inhibitors include Pexidartinib (PLX-3397), Tap et al, N Engl J Med, 373:428-437 (2015); gilteritinib (ASP2215), Smith et al., Blood: 126 (23) (2015); FLX-925, also known as AMG-925, Li et al. Mol. Cancer Ther. 14: 375-83 (2015); and G-749, Lee et al, Blood. 123: 2209-2219 (2014).

In some embodiments, the chemotherapeutic agent is an IDH (isocitrate dehydrogenase) inhibitor. In some embodiments, the IDH inhibitor is a member of the oxazolidinone (3-pyrimidinyl-4-yl-oxazolidin-2-one) family, and is a specific inhibitor of the neomorphic activity of IDH1 mutants and has the chemical name (S)-4-isopropyl-3-(2-(((S)-1-(4 phenoxyphenyl)ethyl)amino)pyrimidin-4-yl)oxazolidin-2-one.

As used herein, the term “adrenomedullin” or “ADM” has its general meaning in the art and refers to which comprises 52 amino acids and which comprises the amino acids 95 to 146 of pre-proADM, from which it is formed by proteolytic cleavage. An exemplary amino acid sequence of CALCRL is represented by SEQ ID NO:1.

>HUMAN ADM OS = Homo sapiens
SEQ ID NO: 1
YRQSMNNFQGLRSFGCRFGTCTVQKLAHQIYQFTDKDKDNVAPRSKISPQ
GY

As used herein the term “CALCRL” has its general meaning in the art and refers to calcitonin receptor like receptor (Gene ID: 10203). CALCRL is also named CRLR or CGRPR. CALCRL is linked to one of three single transmembrane domain receptor activity-modifying proteins (RAMPs) that are essential for functional activity. The association of CALCRL with different RAMP proteins produces different receptors: i) with RAMP1: produces a CGRP receptor, ii) with RAMP2: produces an adrenomedullin (ADM) receptor, designated AM1, and iii) with RAMP3: produces a dual CGRP/ADM receptor designated AM2. These receptors are linked to the G protein Gs which activates adenylate cyclase and activation results in the generation of intracellular cyclic adenosine monophosphate (cAMP). An exemplary amino acid sequence of CALCRL is represented by SEQ ID NO:2. The extracellular domain of CALCR ranges from the amino at position 23 to the amino acid at position 146 in SEQ ID NO:2.

>sp|Q16602|CALRL_HUMAN Calcitonin gene-related
peptide type 1 receptor OS = Homo sapiens
OX = 9606 GN = CALCRL PE = 1 SV = 2
SEQ ID NO: 2
MEKKCTLNFLVLLPFFMILVTAELEESPEDSIQLGVTRNKIMTAQYECYQ
KIMQDPIQQAEGVYCNRTWDGWLCWNDVAAGTESMQLCPDYFQDFDPSEK
VTKICDQDGNWFRHPASNRTWTNYTQCNVNTHEKVKTALNLFYLTIIGHG
LSIASLLISLGIFFYFKSLSCQRITLHKNLFFSFVCNSVVTIIHLTAVAN
NQALVATNPVSCKVSQFIHLYLMGCNYFWMLCEGIYLHTLIVVAVFAEKQ
HLMWYYFLGWGFPLIPACIHAIARSLYYNDNCWISSDTHLLYIIHGPICA
ALLVNLFFLLNIVRVLITKLKVTHQAESNLYMKAVRATLILVPLLGIEFV
LIPWRPEGKIAEEVYDYIMHILMHFQGLLVSTIFCFFNGEVQAILRRNWN
QYKIQFGNSFSNSEALRSASYTVSTISDGPGYSHDCPSEHLNGKSIHDIE
NVLLKPENLYN

As used herein, the expression “inhibitor of adrenomedullin activity or expression” refers to a molecule that partially or fully blocks, inhibits, or neutralizes a biological activity or expression of adrenomedullin. Thus the inhibitor can be a molecule of any type that interferes with the signaling associated with adrenomedullin in leukemic cells, for example, either by decreasing transcription or translation of adrenomedullin-encoding nucleic acid, or by inhibiting or blocking adrenomedullin activity, or both. In particular, the inhibitor inhibits the interaction between adrenomedullin and its receptor CALCRL. Examples of inhibitors include, but are not limited to, antisense polynucleotides, interfering RNAs, catalytic RNAs, RNA-DNA chimeras, aptamers, polypeptides and antibodies.

In some embodiments, the inhibitor is a polypeptide comprising a functional equivalent of CALCRL. For instance, functional equivalents include molecules that bind to adrenomedullin and comprise all or a portion of the extracellular domains of CALCRL so as to form a soluble receptor that is capable to trap adrenomedullin. Accordingly the present invention provides a polypeptide capable of inhibiting binding of CALCRL to Adrenomedullin, which polypeptide comprises consecutive amino acids having a sequence which corresponds to the sequence of at least a portion of an extracellular domain of CALCRL, which portion binds to adrenomedullin. In some embodiments, the polypeptide comprises a functional equivalent of CALCRL which is fused to an immunoglobulin constant domain (Fc region) to form an immunoadhesin. Immunoadhesins can possess many of the valuable chemical and biological properties of human antibodies. Since immunoadhesins can be constructed from a human protein sequence with a desired specificity linked to an appropriate human immunoglobulin hinge and constant domain (Fc) sequence, the binding specificity of interest can be achieved using entirely human components. The immunoglobulin sequence typically, but not necessarily, is an immunoglobulin constant domain. The immunoglobulin moiety in the chimeras of the present invention may be obtained from IgG1, IgG2, IgG3 or IgG4 subtypes, IgA, IgE, IgD or IgM, but typically IgG1 or IgG3. In some embodiments, the functional equivalent of the PD-1 or CALCRL and the immunoglobulin sequence portion of the immunoadhesin are linked by a minimal linker.

In some embodiments, the inhibitor is an antibody that binds to adrenomedullin. In some embodiments, the antibody binds to at least 2, 3, 4, 5, 6, 7, 8, 9, 10, 11 consecutive amino acids located in the sequence which ranges from the amino acid residue at position 42 to the amino acid residue at position 52 in SEQ ID NO:1. In some embodiments, the antibody binds to the the sequence which ranges from the amino acid residue at position 42 to the amino acid residue at position 52 in SEQ ID NO:1.

As used herein, the term “antibody” is thus used to refer to any antibody-like molecule that has an antigen binding region, and this term includes antibody fragments that comprise an antigen binding domain such as Fab′, Fab, F(ab′)2, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa (lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art (see Kabat et al., 1991, specifically incorporated herein by reference). Diabodies, in particular, are further described in EP 404, 097 and WO 93/1 1 161; whereas linear antibodies are further described in Zapata et al. (1995). Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art. For example, each of Beckman et al., 2006; Holliger & Hudson, 2005; Le Gall et al., 2004; Reff & Heard, 2001; Reiter et al., 1996; and Young et al., 1995 further describe and enable the production of effective antibody fragments.

In natural antibodies, two heavy chains are linked to each other by disulfide bonds and each heavy chain is linked to a light chain by a disulfide bond. There are two types of light chain, lambda (l) and kappa (k). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) influence the overall domain structure and hence the combining site. Complementarity Determining Regions or CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, includes six CDRs, comprising the CDR set from each of a heavy and a light chain V region. Framework Regions (FRs) refer to amino acid sequences interposed between CDRs.

As used herein the term “bind” indicates that the antibody has affinity for the surface molecule. The term “affinity”, as used herein, means the strength of the binding of an antibody to an epitope. The affinity of an antibody is given by the dissociation constant Kd, defined as [Ab]×[Ag]/[Ab-Ag], where [Ab-Ag] is the molar concentration of the antibody-antigen complex, [Ab] is the molar concentration of the unbound antibody and [Ag] is the molar concentration of the unbound antigen. The affinity constant Ka is defined by 1/Kd. Preferred methods for determining the affinity of mAbs can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One preferred and standard method well known in the art for determining the affinity of mAbs is the use of Biacore instruments.

In some embodiments, the antibody of the present invention is a monoclonal antibody. The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, monoclonal antibodies are advantageous in that they can be synthesized by hybridoma cells that are uncontaminated by other immunoglobulin producing cells. Alternative production methods are known to those trained in the art, for example, a monoclonal antibody may be produced by cells stably or transiently transfected with the heavy and light chain genes encoding the monoclonal antibody. Monoclonal antibodies may be generated using the method of Kohler and Milstein (Nature, 256:495, 1975). To prepare monoclonal antibodies useful in the invention, a mouse or other appropriate host animal is immunized at suitable intervals (e.g., twice-weekly, weekly, twice-monthly or monthly) with the appropriate antigenic forms (i.e. polypeptides of the present invention). The animal may be administered a final “boost” of antigen within one week of sacrifice. It is often desirable to use an immunologic adjuvant during immunization. Suitable immunologic adjuvants include Freund's complete adjuvant, Freund's incomplete adjuvant, alum, Ribi adjuvant, Hunter's Titermax, saponin adjuvants such as QS21 or Quil A, or CpG-containing immunostimulatory oligonucleotides. Other suitable adjuvants are well-known in the field. The animals may be immunized by subcutaneous, intraperitoneal, intramuscular, intravenous, intranasal or other routes. A given animal may be immunized with multiple forms of the antigen by multiple routes. Briefly, the recombinant polypeptide of the present invention may be provided by expression with recombinant cell lines. Recombinant forms of the polypeptides may be provided using any previously described method. Following the immunization regimen, lymphocytes are isolated from the spleen, lymph node or other organ of the animal and fused with a suitable myeloma cell line using an agent such as polyethylene glycol to form a hydridoma. Following fusion, cells are placed in media permissive for growth of hybridomas but not the fusion partners using standard methods. Following culture of the hybridomas, cell supernatants are analysed for the presence of antibodies of the desired specificity, i.e., that selectively bind the antigen. Suitable analytical techniques include ELISA, flow cytometry, immunoprecipitation, and western blotting. Other screening techniques are well-known in the field. Preferred techniques are those that confirm binding of antibodies to conformationally intact, natively folded antigen, such as non-denaturing ELISA, flow cytometry, and immunoprecipitation.

In some embodiments, the monoclonal antibody of the invention is a chimeric antibody, in particular a chimeric mouse/human antibody. As used herein, the term “chimeric antibody” refers to an antibody which comprises a VH domain and a VL domain of a non-human antibody, and a CH domain and a CL domain of a human antibody. In some embodiments, the human chimeric antibody of the present invention can be produced by obtaining nucleic sequences encoding VL and VH domains as previously described, constructing a human chimeric antibody expression vector by inserting them into an expression vector for animal cell having genes encoding human antibody CH and human antibody CL, and expressing the coding sequence by introducing the expression vector into an animal cell. As the CH domain of a human chimeric antibody, it may be any region which belongs to human immunoglobulin, but those of IgG class are suitable and any one of subclasses belonging to IgG class, such as IgG1, IgG2, IgG3 and IgG4, can also be used. Also, as the CL of a human chimeric antibody, it may be any region which belongs to Ig, and those of kappa class or lambda class can be used. Methods for producing chimeric antibodies involve conventional recombinant DNA and gene transfection techniques are well known in the art (See Morrison S L. et al. (1984) and patent documents U.S. Pat. Nos. 5,202,238; and 5,204,244).

In some embodiments, the monoclonal antibody of the invention is a humanized antibody. In particular, in said humanized antibody, the variable domain comprises human acceptor frameworks regions, and optionally human constant domain where present, and non-human donor CDRs, such as mouse CDRs. According to the invention, the term “humanized antibody” refers to an antibody having variable region framework and constant regions from a human antibody but retains the CDRs of a previous non-human antibody. The humanized antibody of the present invention may be produced by obtaining nucleic acid sequences encoding CDR domains, as previously described, constructing a humanized antibody expression vector by inserting them into an expression vector for animal cell having genes encoding (i) a heavy chain constant region identical to that of a human antibody and (ii) a light chain constant region identical to that of a human antibody, and expressing the genes by introducing the expression vector into an animal cell. The humanized antibody expression vector may be either of a type in which a gene encoding an antibody heavy chain and a gene encoding an antibody light chain exists on separate vectors or of a type in which both genes exist on the same vector (tandem type). In respect of easiness of construction of a humanized antibody expression vector, easiness of introduction into animal cells, and balance between the expression levels of antibody H and L chains in animal cells, humanized antibody expression vector of the tandem type is preferred. Examples of tandem type humanized antibody expression vector include pKANTEX93 (WO 97/10354), pEE18 and the like. Methods for producing humanized antibodies based on conventional recombinant DNA and gene transfection techniques are well known in the art (See, e.g., Riechmann L. et al. 1988; Neuberger M S. et al. 1985). Antibodies can be humanized using a variety of techniques known in the art including, for example, CDR-grafting (EP 239,400; PCT publication WO91/09967; U.S. Pat. Nos. 5,225,539; 5,530,101; and 5,585,089), veneering or resurfacing (EP 592,106; EP 519,596; Padlan E A (1991); Studnicka G M et al. (1994); Roguska M A. et al. (1994)), and chain shuffling (U.S. Pat. No. 5,565,332). The general recombinant DNA technology for preparation of such antibodies is also known (see European Patent Application EP 125023 and International Patent Application WO 96/02576).

In some embodiments the antibody of the invention is a human antibody. As used herein the term “human antibody is intended to include antibodies having variable and constant regions derived from human immunoglobulin sequences. The human antibodies of the present invention may include amino acid residues not encoded by human immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences. Human antibodies can be produced using various techniques known in the art. Human antibodies are described generally in van Dijk and van de Winkel, cur. Opin. Pharmacol. 5; 368-74 (2001) and lonberg, cur. Opin.Immunol. 20; 450-459 (2008). Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat.Biotech. 23; 1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. patent application publication No. US 2007/0061900, describing VELOCIMOUSE® technology. Human variable regions from intact antibodies generated by such animals may be further modified, e.g., by combining with a different human constant region Human antibodies can also be made by hybridoma-based methods. Human myeloma and mouse-human heteromyeloma cell lines for the production of human monoclonal antibodies have been described. (See, e.g., Kozbor J. Immunol., 13: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human igM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005). Fully human antibodies can also be derived from phage-display libraries (as disclosed in Hoogenboom et al., 1991, J. Mol. Biol. 227:381; and Marks et al., 1991, J. Mol. Biol. 222:581). Phage display techniques mimic immune selection through the display of antibody repertoires on the surface of filamentous bacteriophage, and subsequent selection of phage by their binding to an antigen of choice. One such technique is described in PCT publication No. WO 99/10494. Human antibodies described herein can also be prepared using SCID mice into which human immune cells have been reconstituted such that a human antibody response can be generated upon immunization. Such mice are described in, for example, U.S. Pat. Nos. 5,476,996 and 5,698,767 to Wilson et al.

In some embodiments, the antibody of the present invention is one antibody disclosed in WO2013072510 or in Struck J, Hein F, Karasch S, Bergmann A. Epitope specificity of anti-Adrenomedullin antibodies determines efficacy of mortality reduction in a cecal ligation and puncture mouse model. Intensive Care Med Exp. 2013; 1(1):22. doi:10.1186/2197-425X-1-3.

In some embodiments, the inhibitor is an inhibitor of adrenomedullin expression. An “inhibitor of expression” refers to a natural or synthetic compound that has a biological effect to inhibit the expression of a gene. In a preferred embodiment of the invention, said inhibitor of gene expression is a siRNA, an antisense oligonucleotide or a ribozyme. For example, anti-sense oligonucleotides, including anti-sense RNA molecules and anti-sense DNA molecules, would act to directly block the translation of the mRNA encoding for the precursor of adrenomedullin by binding thereto and thus preventing protein translation or increasing mRNA degradation, thus decreasing the level of adrenomedullin, and thus activity, in a cell. For example, antisense oligonucleotides of at least about 15 bases and complementary to unique regions of the mRNA transcript sequence encoding the precursor of adrenomedullin can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). Small inhibitory RNAs (siRNAs) can also function as inhibitors of expression for use in the present invention. the precursor of adrenomedullin gene expression can be reduced by contacting a patient or cell with a small double stranded RNA (dsRNA), or a vector or construct causing the production of a small double stranded RNA, such that the precursor of adrenomedullin gene expression is specifically inhibited (i.e. RNA interference or RNAi). Antisense oligonucleotides, siRNAs, shRNAs and ribozymes of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and typically cells expressing the precursor of adrenomedullin. Typically, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art. In some embodiments, the inhibitor of expression is an endonuclease. The term “endonuclease” refers to enzymes that cleave the phosphodiester bond within a polynucleotide chain. Some, such as Deoxyribonuclease I, cut DNA relatively nonspecifically (without regard to sequence), while many, typically called restriction endonucleases or restriction enzymes, and cleave only at very specific nucleotide sequences. The mechanism behind endonuclease-based genome inactivating generally requires a first step of DNA single or double strand break, which can then trigger two distinct cellular mechanisms for DNA repair, which can be exploited for DNA inactivating: the errorprone nonhomologous end-joining (NHEJ) and the high-fidelity homology-directed repair (HDR). In a particular embodiment, the endonuclease is CRISPR-cas. As used herein, the term “CRISPR-cas” has its general meaning in the art and refers to clustered regularly interspaced short palindromic repeats associated which are the segments of prokaryotic DNA containing short repetitions of base sequences. In some embodiment, the endonuclease is CRISPR-cas9 which is from Streptococcus pyogenes. The CRISPR/Cas9 system has been described in U.S. Pat. No. 8,697,359 B1 and US 2014/0068797. In some embodiment, the endonuclease is CRISPR-Cpf1 which is the more recently characterized CRISPR from Provotella and Francisella 1 (Cpf1) in Zetsche et al. (“Cpf1 is a Single RNA-guided Endonuclease of a Class 2 CRISPR-Cas System (2015); Cell; 163, 1-13).

By a “therapeutically effective amount” is meant a sufficient amount of the inhibitor at a reasonable benefit/risk ratio applicable to the medical treatment. It will be understood that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

Typically, the inhibitor of the present invention is combined with pharmaceutically acceptable excipients, and optionally sustained-release matrices, such as biodegradable polymers, to form pharmaceutical compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. Typically, the pharmaceutical compositions contain vehicles, which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Sterile injectable solutions are prepared by incorporating the active ingredient at the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The invention will be further illustrated by the following figures and examples. However, these examples and figures should not be interpreted in any way as limiting the scope of the present invention.

### FIGURES

FIG. 1. Expression of Adrenomedullin and Impact on Patient Outcome in AML

(A) Overall survival and Event-Free Survival according to ADM H-scores. (B) Overall and Event-Free Survival according to CALCRL and ADM H-scores. *p<0.05; **p<0.01; ***p<0.001; ns, not significant.

FIG. 2. Depletion of ADM Impairs Leukemic Cell Growth.

(A) Western blot results showing expression of ADM and 3-ACTIN proteins in MOLM-14 and OCI-AML3 four days after transduction with shADM. (B) Graph shows cell number of MOLM-14 or OCI-AML3. Three days after transduction, cells were plated at 0.3M cells per ml (DO) and cell proliferation was followed using trypan blue exclusion. (C) Graph shows the percentage of Annexin-V+ or 7-AAD+ cells 4 days after cell transduction.

FIG. 3. Depletion of AM Sensitizes to Chemotherapeutic Drugs.

Graph shows the percentage of Annexin-V+ or 7-AAD+ cells. Three days after transduction with shADM.

FIG. 4. Chemotherapy Reduced Both Percentage of Human Cells and Levels of Secreted ADM in the Bone Marrow of Mice.

(A) Percentage of human cells in the murine bone marrow in PBS and AraC-treated mice. (B) Western-Blot and graph showing the protein expression of ADM in the bone marrow supernatant of xenografted mice treated with PBS or AraC.

### EXAMPLE

Methods

Human Studies

Primary AML patient specimens are from Toulouse University Hospital (TUH), Toulouse, France]. Frozen samples were obtained from patients diagnosed with AML at TUH after signed informed consent in accordance with the Declaration of Helsinki, and stored at the HIMIP collection (BB-0033-00060). According to the French law, HIMIP biobank collection has been declared to the Ministry of Higher Education and Research (DC 2008-307, collection 1) and obtained a transfer agreement (AC 2008-129) after approbation by the Comité de Protection des Personnes Sud-Ouest et Outremer II (ethical committee). Clinical and biological annotations of the samples have been declared to the CNIL (Comité National Informatique et Libertés ie Data processing and Liberties National Committee). See Table S3 for age, sex, cytogenetics and mutation information on human specimens used in the current study.

In vivo animal studies NSG (NOD.Cg-Prkdcscid Il2rgtmlWjI/SzJ) mice (Charles River Laboratories) were used for transplantation of AML cell lines or primary AML samples. Male or Female mice ranging in age from 6 to 9 weeks were started on experiment and before cell injection or drug treatments, mice were randomly assigned to experimental groups. Mice were housed in sterile conditions using HEPA-filtered micro-isolators and fed with irradiated food and sterile water in the Animal core facility of the Cancer Research Center of Toulouse (France). All animals were used in accordance with a protocol reviewed and approved by the Institutional Animal Care and Use Committee of Region Midi-Pyrenees (France).

Cell lines and primary cultures For primary human AML cells, peripheral blood or bone marrow samples were frozen in FCS with 10% DMSO and stored in liquid nitrogen. The percentage of blasts was determined by flow cytometry and morphologic characteristics before purification. Cells were thawed in 37° C. water bath, washed in thawing media composed of IMDM, 20% FBS. Then cells were maintained in IMDM, 20% FBS and 1% Pen/Strep (GIBCO) for all experiments.

Cell Lines and Culture Conditions

Human AML cell lines were maintained in RPMI-media (Gibco) supplemented with 10% FBS (Invitrogen) in the presence of 100 U/mL of penicillin and 100 g/mL of streptomycin, and were incubated at 37° C. with 5% CO2. The cultured cells were split every 2 to 3 days and maintained in an exponential growth phase. All AML cell lines were purchased at DSMZ or ATCC, and their liquid nitrogen stock were renewed every 2 years. These cell lines have been routinely tested for Mycoplasma contamination in the laboratory. The U937 cells were obtained from the DSMZ in February 2012 and from the ATCC in January 2014. MV4-11 and HL-60 cells were obtained from the DSMZ in February 2012 and 2016. KG1 cells were obtained from the DSMZ in February 2012 and from the ATCC in March 2013. KG1a cells were obtained from the DSMZ in February 2016. MOLM14 was obtained from Pr. Martin Carroll (University of Pennsylvania, Philadelphia, Pa.) in 2011.

Mouse Xenograft Model

NSG mice were produced at the Genotoul Anexplo platform at Toulouse (France) using breeders obtained from Charles River Laboratories. Transplanted mice were treated with antibiotic (Baytril) for the duration of the experiment. For experiments assessing the response to chemotherapy in PDX models, mice (6-9 weeks old) were sublethally treated with busulfan (30 mg/kg) 24 hours before injection of leukemic cells. Leukemia samples were thawed in 37° C. water bath, washed in IMDM 20% FBS, and suspended in Hank's Balanced Salt Solution at a final concentration of 1-10×106 cells per 200 μL for tail vein injection in NSG mice. Eight to 18 weeks after AML cell transplantation and when mice were engrafted (tested by flow cytometry on peripheral blood or bone marrow aspirates), NSG mice were treated by daily intraperitoneal injection of 60 mg/kg AraC or vehicle (PBS) for 5 days. AraC was kindly provided by the pharmacy of the TUH. Mice were sacrificed at day 8 to harvest human leukemic cells from murine bone marrow. For AML cell lines, 24 hours before injection of leukemic cells mice were treated with busulfan (20 mg/kg). Then cells were thawed and washed as previously described, suspended in HBSS at a final concentration of 2×106 per 200 μL before injection into bloodstream of NSG mice. For experiments using inducible shRNAs, doxycycline (0.2 mg/ml+1% sucrose) was added to drinking water the day of cell injection or 10 days after until the end of the experiment. Mice were treated by daily intraperitoneal injection of 30 mg/kg AraC for 5 days and sacrificed at day 8. Daily monitoring of mice for symptoms of disease (ruffled coat, hunched back, weakness, and reduced mobility) determined the time of killing for injected animals with signs of distress.

Assessment of Leukemic Engraftment

At the end of experiment, NSG mice were humanely killed in accordance with European ethics protocols. Bone marrow (mixed from tibias and femurs) and spleen were dissected and flushed in HBSS with 1% FBS. MNCs from bone marrow, and spleen were labeled with anti-hCD33, anti-mCD45.1, anti-hCD45, anti-hCD3 and/or anti-hCD44 (all from BD) antibodies to determine the fraction of viable human blasts (hCD3-hCD45+mCD45.1-hCD33+/hCD44+AnnV-cells) using flow cytometry. In some experiments, we also added anti-CALCRL, anti-CD34 and anti-CD38 to characterize AML stem cells. Monoclonal antibody recognizing extracellular domain of CALCRL was generated in the lab with the help of Biotem company (France). Then antibody was labelled with R-Phycoerythrin using Lightning-Link kit (Expedeon). All antibodies were used at concentrations between 1/50 and 1/200 depending on specificity and cell density. Analyses were performed on a LSRFortessa flow cytometer with DIVA software (BD Biosciences) or CytoFLEX flow cytometer with CytoExpert software (Beckman Coulter). The number of AML cells/μL peripheral blood and number of AML cells in total cell tumor burden (in bone marrow and spleen) were determined by using CountBright beads (Invitrogen) using described protocol.

For LDA experiments, human engraftment was considered positive if at least >0.1% of cells in the murine bone marrow were hCD45+mCD45.1-hCD33+. The cut-off was increased to >0.5% for AML #31 because the engraftment was measured only based on hCD45+mCD45.1−. Limiting dilution analysis was performed using ELDA software.

Western Blot Analysis

Proteins were resolved using 4% to 12% polyacrylamide gel electrophoresis Bis-Tris gels (Life Technology, Carlsbad, Calif.) and electrotransferred to nitrocellulose membranes. After blocking in Tris-buffered saline (TBS) 0.1%, Tween 20%, 5% bovine serum albumin, membranes were immunostained overnight with appropriate primary antibodies followed by incubation with secondary antibodies conjugated to HRP. Immunoreactive bands were visualized by enhanced chemiluminescence (ECL Supersignal West Pico; Thermo Fisher Scientific) with a Syngene camera. Quantification of chemiluminescent signals was done with the GeneTools software from Syngene.

Cell Death Assay

After treatment, 5.105 cells were washed with PBS and resuspended in 200 L of Annexin-V binding buffer (BD biosciences). Two microliters of Annexin-V-FITC (BD Biosciences) and 7-amino-actinomycin D (7-AAD; Sigma Aldrich) were added for 15 minutes at room temperature in the dark. All samples were analyzed using LSRFortessa or CytoFLEX flow cytometer.

Cell Cycle Analysis

Cells were harvested, washed with PBS and fixed in ice-cold 70% ethanol at −20° C. Cells were then permeabilized with 1×PBS containing 0.25% Triton X-100, resuspended in 1×PBS containing 10 μg/ml propidium iodide and 1 μg/ml RNase, and incubated for 30 min at 37° C. Data were collected on a CytoFLEX flow cytometer.

Clonogenic Assay

Primary cells from AML patients were thawed and resuspended in 100 μl Nucleofector Kit V (Amaxa, Cologne, Germany). Then, cells were nucleofected according to the manufacturer's instructions (program U-001 Amaxa, Cologne, Germany) with 200 nM siRNA scrambled (ON-TARGETplus Non-targeting siRNA #2, Dharmacon) or anti-CALCRL (SMARTpool ON-TARGETplus CALCRL siRNA, Dharmacon). Cells were adjusted to 1×105 cells/ml final concentration in H4230 methylcellulose medium (STEMCELL Technologies) supplemented with 10% 5637-CM as a stimulant and then plated in 35-mm petri dishes in duplicate and allowed to grow for 7 days in a humidified CO2 incubator (5% CO2, 37° C.). At day 7, the leukemic colonies (more than five cells) were scored.

shRNA, Lentiviral Production and Leukemic Cell Transduction

shRNA sequences were constructed into pLKO-TET-ON or bought cloned into pLKO vectors. Each construct (6 μg) was co-transfected using lipofectamine 2000 (20 μL) in 10 cm-dish with psPax2 (4 μg, provides packaging proteins) and pMD2.G (2 μg, provides VSV-g envelope protein) plasmids into 293T cells to produce lentiviral particles. Twenty-four hours after cell transfection, medium was removed and 10 ml opti-MEM+1% Pen/Strep was added. At about 72 hours post transfection, 293T culture supernatants containing lentiviral particles were harvested, filtered, aliquoted and stored in −80° C. freezer for future use. At the day of transduction, cells were infected by mixing 2.106 cells in 2 ml of freshly thawed lentivirus and Polybrene at a final concentration of 8 ug/ml. At 3 days post infection, transduced cells were selected using 1 μg/ml puromycin.

EC50 Experiments

The day before experiment, cells were adjusted to 3×105 cells/ml final concentration and plated in a 96-well plate (final volume: 100 l). To measure half-maximal inhibitory concentration (EC50), increased concentrations of AraC or idarubicin were added to the medium. After two days, 20 l per well of MTS solution (Promega) was added for two hours and then absorbance was recorded at 490 nm with a 96-well plate reader. The doses that decrease cell viability to 50% (EC50) were analyzed Nonlinear regression log [inhibitor] vs. normalized response-variable slope with GraphPad Prism software.

Measurement of oxygen consumption in AML cultured cells using Seahorse Assay All XF assays were performed using the XFp Extracellular Flux Analyser (Seahorse Bioscience, North Billerica, Mass.). The day before the assay, the sensor cartridge was placed into the calibration buffer medium supplied by Seahorse Biosciences to hydrate overnight. Seahorse XFp microplates wells were coated with 50 μl of Cell-Tak (Corning; Cat #354240) solution at a concentration of 22.4 μg/ml and kept at 4° C. overnight. Then, Cell-Tak coated Seahorse microplates wells were rinsed with distillated water and AML cells were plated at a density of 105 cells per well with XF base minimal DMEM media containing 11 mM glucose, 1 mM pyruvate and 2 mM glutamine. Then, 180 μl of XF base minimal DMEM medium was placed into each well and the micrcoplate was centrifuged at 80 g for 5 min. After one hour incubation at 37° C. in CO2 free-atmosphere, basal oxygen consumption rate (OCR, as a mitochondrial respiration indicator) and extracellular acidification rate (ECAR, as a glycolysis indicator) were performed using the XFp analyzer.

RNA Microarray and Bioinformatics Analyses

For primary AML samples, human CD45+CD33+ were isolated using cell sorter cytometer from engrafted BM mice (for 3 primary AML specimens) treated with PBS or treated with AraC. RNA from AML cells was extracted using Trizol (Invitrogen) or RNeasy (Qiagen). For MOLM-14 AML cell line, mRNA from 2.106 of cells was extracted using RNeasy (Qiagen). RNA purity was monitored with NanoDrop 1ND-1000 spectrophotometer and RNA quality was assessed through Agilent 2100 Bionalyzer with RNA 6000 Nano assay kit. No RNA degradation or contamination were detected (RIN>9). 100 ng of total RNA were analysed on Affymetrix GeneChip© Human Gene 2.0 ST Array using the Affymetrix GeneChip© WT Plus Reagent Kit according to the manufacturer's instructions (Manual Target Preparation for GeneChip® Whole Transcript (WT) Expression Arrays P/N 703174 Rev. 2). Arrays were washed and scanned; and the raw files generated by the scanner was transferred into R software for preprocessing (with RMA function, Oligo package), quality control (boxplot, clustering and PCA) and differential expression analysis (with eBayes function, LIMMA package). Prior to differential expression analysis, all transcript clusters without any gene association were removed. Mapping between transcript clusters and genes were done using annotation provided by Affymetrix (HuGene-2_0-st-v1.na36.hg19.transcript.csv) and the R/Bioconductor package hugene20sttranscriptcluster.db. p-values generated by the eBayes function were adjusted to control false discovery using the Benajmin and Hochberg's procedure. [RMA] Irizarry et al., Biostatistics, 2003; [Oligo package] Carvalho and Irizarry, Bioinformatics, 2010; [LIMMA reference] Ritchie et al., Nucleic Acids Research, 2015; hugene20sttranscriptcluster.db.MacDonald J W 2017, Affymetrix hugene20 annotation data (chip hugene20sttranscriptcluster); [FDR]: Benjamini et al., Journal of the Royal Statistical Society, 1995.

GSEA Analysis

GSEA analysis was performed using GSEA version 3.0 (Broad Institute). Gene signatures used in this study were from Broad Institute database, literature, or in-house built. Following parameters were used: Number of permutations=1000, permutation type=gene_set. Other parameters were left at default values.

Quantification and Statistical Analysis

We assessed the statistical analysis of the difference between two sets of data using two-tailed (non-directional) Student's t test with Welch's correction. For survival analyses, we used Log-rank (Mantel-Cox) test. For Limit Dilution Assay experiments, frequency and statistics analyses were performed using L-calc software (Stemcell technologies). A p value of less than 0.05 indicates significance. *p<0.05; **p<0.01; ***p<0.001; ****p<0.0001; ns, not significant. Detailed information of each test is in the figure legends.

All publicly accessible transcriptomic databases of AML patients used in this study: GSE30377: Eppert K, Takenaka K, Lechman E R, Waldron L, Nilsson B, van Galen P, Metzeler K H, Poeppl A, Ling V, Beyene J, Canty A J, Danska J S, Bohlander S K, Buske C, Minden M D, Golub T R, Jurisica I, Ebert B L, Dick J E. (28 Aug. 2011) Stem cell gene expression programs influence clinical outcome in human leukemia. Nat Med, 17(9), 1086-93.

GSE14468: Verhaak R G, Wouters B J, Erpelinck C A, Abbas S, Beverloo H B, Lugthart S, Lowenberg B, Delwel R, Valk P J. (January 2009) Prediction of molecular subtypes in acute myeloid leukemia based on gene expression profiling. Haematologica, 94(1), 131-4.

GSE12417: Metzeler K H, Hummel M, Bloomfield C D, Spiekermann K, Braess J, Sauerland M C, Heinecke A, Radmacher M, Marcucci G, Whitman S P, Maharry K, Paschka P, Larson R A, Berdel W E, Büchner T, Wörmann B, Mansmann U, Hiddemann W, Bohlander S K, Buske C; Cancer and Leukemia Group B; German A ML Cooperative Group. (15 Nov. 2008) An 86-probe-set geneexpression signature predicts survival in cytogenetically normal acute myeloid leukemia. Blood, 112(10), 4193-201.

GSE116256: Van Galen P, Hovestadt V, Wadsworth Ii M H, Hughes T K, Griffin G K, Battaglia S, Verga J A, Stephansky J, Pastika T J, Lombardi Story J, Pinkus G S, Pozdnyakova O, Galinsky I, Stone R M, Graubert T A, Shalek A K, Aster J C, Lane A A, Bernstein B E. Single-cell RNA-seq reveals AML hierarchies relevant to disease progression and immunity. Cell. 2019 March 7; 176(6):1265-1281.

TCGA: The Cancer Genome Atlas Research Network. (30 May 2013) Genomic and epigenomic landscapes of adult de novo acute myeloid leukemia. N Engl J Med, 368(22), 2059-74. Erratum in: N Engl J Med. 2013 Jul. 4; 369(1):98.

Results

The Receptor CALCRL and its Ligand Adrenomedullin are Expressed in AML Cells and Associated with a Poor Outcome in Patients

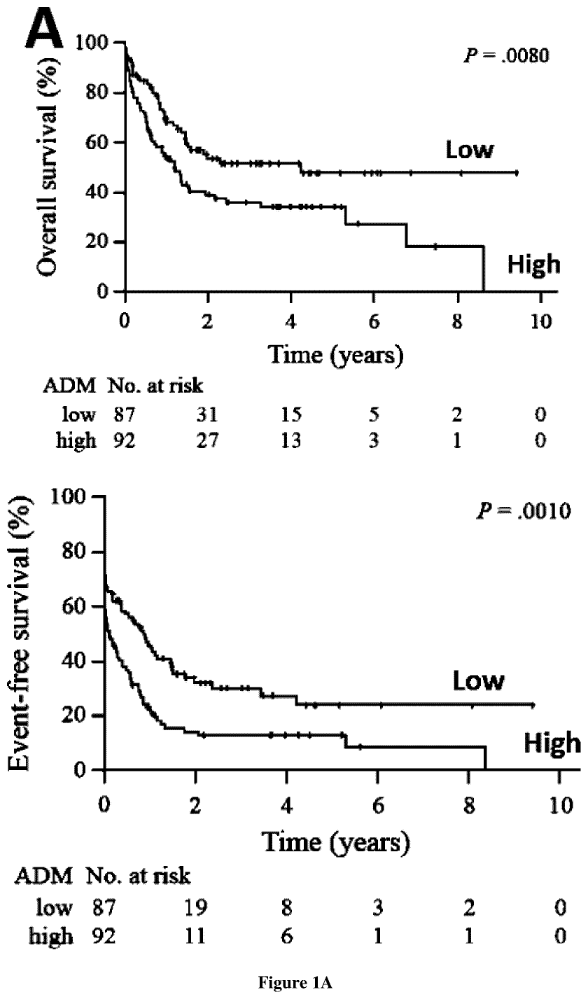

Using a clinically relevant chemotherapeutic model, we and others previously demonstrated that LSCs are not necessarily enriched in AraC residual AML, suggesting that these cells are also targeted by chemotherapy and LSCs are comprised of both chemosensitive and chemoresistant stem cell sub-populations (Farge et al., 2017; Boyd et al., 2018). In order to identify chemoresistant LSCs which are at the origin of AML regeneration after chemotherapy, we analyzed transcriptomic data from three different studies that (data not shown): i) identified 134 genes overexpressed in functionally defined LSC compared with a normal HSC counterpart (Eppert et al., 2011; GSE30377); ii) uncovered 114 genes of high expression associated with poor prognosis in AML (the Cancer Genome Atlas, AML cohort, 2013); and iii) selected 536 genes overexpressed at relapse compared to pairwise matched diagnosis after intensive chemotherapy (Hackl et al., 2015; GSE66525). Surprisingly, we found one unique gene common to these three independent transcriptomic databases: CALCRL encoding for a G protein-coupled seven-transmembrane domain receptor poorly documented in cancer that has been recently described as associated with bad prognosis in AML (Angenendt et al, 2019). Using three independently published cohorts of AML patients (TCGA AML cohort; GSE12417; GSE14468), we confirmed that patients with high CALCRL expression had a worse overall survival (data not shown) and are more refractory to chemotherapy (data not shown) compared to patients with low CALCRL expression. This is correlated with a greater expression in complex and normal karyotypes compared with Core Binding Factor AML (CBF) (data not shown). Furthermore, CALCRL gene expression was significantly higher at relapse compared to diagnosis in patients treated with intensive chemotherapy (data not shown). CALCRL expression was also higher in the leukemic compartment compared with normal hematopoietic cells, and more specifically in the LSC population as both functionally—(data not shown) and phenotypically—(data not shown) defined compared with the AML bulk population. Interestingly, CALCRL expression negatively correlated with increasing FAB type, suggesting that CALCRL is a marker of cell immaturity (data not shown). Then, we confirmed by flow cytometry analysis that cell surface expression of CALCRL is higher in the leukemic bulk (n=37) vs. normal bulk (n=9) population (Fold change, FC=2.3; data not shown) Next, we assessed the expression of ADM, a CALCRL ligand already described in several cancers (Berenguer-Daizé et al., 2013; Kocemba et al., 2013). TheADMgene is overexpressed in AML cells compared to normal cells (data not shown) although its expression is not altered in AML patients at relapse compared to initial diagnosis (data not shown). Using a combination of western blotting, confocal microscopy and RNA microarray, we have established that CALCRL, its three co-receptors RAMP1, RAMP2 and RAMP3, ADM (but not CGRP, another putative CALCRL ligand) are expressed in all tested AML cell lines and primary AML samples (data not shown). Then we addressed the impact of CALCRL and ADM protein level on patient outcome. Using IHC analyses, we observed that increasing protein levels of CALCRL or ADM (FIG. 1A) were associated with decreasing complete remission rates, 5-year overall, and event-free survival in a cohort of 198 AML patients. When patients are clustered into 4 groups according to CALCRL and ADM expression (FIG. 1B), we observed that the CALCRL^{high}/ADM^{high} group had a strong reduction of overall survival and that the high expression of only CALCRL or ADM is sufficient to dramatically reduce EFS and complete remission rate (FIG. 1B). All these data supported the hypothesis that the ADM-CALCRL axis is activated in an autocrine fashion in AML and related to a poor prognosis.

The CALCRL-ADM Axis is Required for Cell Growth and Survival

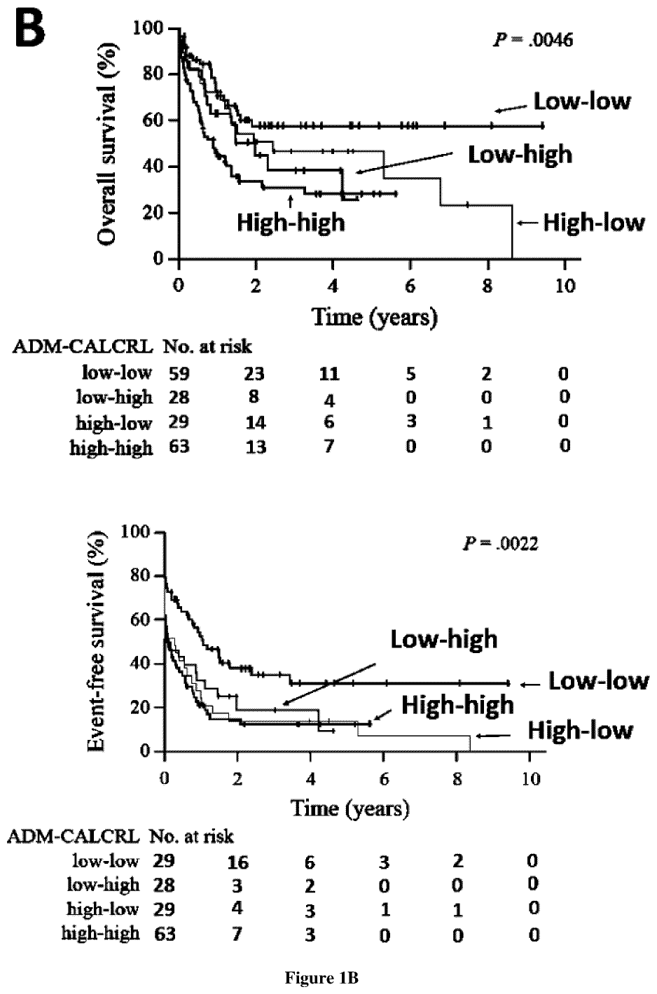

Given the role of several GPCRs identified in AML such as CXCR4 or GPR56 on cell survival and proliferation (Chen et al., 2013; Daria et al., 2016; Pabst et al., 2016), we then investigated whether the CALCRL-ADM axis had an impact on these properties. CALCRL depletion was associated with a decrease of blast cell proliferation (data not shown), an increase in cell death (data not shown) in three AML (MOLM-14, OCI-AML2, OCI-AML3) cell lines. Furthermore, ADM-targeting shRNA (FIG. 2A) phenocopied the effects of shCALCRL on cell proliferation and apoptosis in MOLM-14 and OCI-AML3 cells (FIG. 2B-C). In order to confirm these results in vivo and to control the invalidation of the target over time, we have developed tetracycline-inducible shRNA models. First, we established that the inducible depletion of CALCRL was associated with a decrease in cell proliferation and an increase in apoptosis as observed with constitutive shRNA approaches (data not show). After injection of AML cells in mice, RNA depletion was activated from the first day by doxycycline (data not show). Twenty-five days post-transplantation, the engraftment of human leukemic cells from murine bone marrow and spleen was assessed with mCD45.1⁻hCD45⁺hCD33⁺AnV⁻ markers (data not show). Mice injected with shCAL #1 and shCAL #2 had a significant reduction in AML blasts versus shCTR in both MOLM-14 (shCTR=13.9M vs shCAL #1=0.3 M vs shCAL #2=0.1M; data not show) and OCI-AML3 cells (shCTR=17.2M vs shCAL #1=2.0 M vs shCAL #2=1.7M; data not show). Accordingly, this reduction in total cell tumor burden (TCTB) led to a preservation of the murine bone marrow (data not show). Finally, CALCRL silencing significantly prolonged mice survival (data not show). To take full advantage of our inducible constructs and to improve the clinical relevance of our model, we assessed the impact of CALCRL depletion on established disease (data not show). Short hairpin RNA expression was induced 10 days post-transplantation of shCTR or shCAL in MOLM14 cells after verifying that the level of engraftment was similar in both groups (data not show). In this established disease model, we observed a marked reduction in bone marrow blasts in the mice xenografted with shCAL AML cells compared to the shCTR-xenografted mice cohort in which the disease progressed (data not show). Furthermore, CALCRL downregulation significantly increased mice survival (data not show). Importantly, these results demonstrated that the reduction of blast number and the increase in mice survival observed after CALCRL depletion was not the consequence of an inhibition of blast homing in the murine bone marrow. Altogether, we demonstrated that CALCRL is required for the propagation as well as the maintenance of AML cells in vivo.

CALCRL is Required for Leukemic Stem Cell Maintenance

As CALCRL expression is linked to an immature phenotype and CALCRL depletion impaired AML growth in cell lines, we next aimed to address the role of CALCRL in LSC biology. First, we analyzed previously published single cell RNA-sequencing data (van Galen et al., 2019) and observed that CALCRL is preferentially expressed in HSC-like and progenitor-like cells (Prog-like cells compared with more committed cells in 11 AML patients (data not show). Moreover, while HSC-like and Prog-like cells represent only 34.3% of the total of leukemic cells found in these patients, they accounted for more than 80% of CALCRL^{positive} cells (data not show). Gene Set Enrichment Analysis (GSEA) confirmed that several LSC-associated gene signatures (Eppert et al., 2011; Gentles, 2010; Ng et al., 2016) (data not show) are significantly enriched in AML patients (the Cancer Genome Atlas, AML cohort, 2013) exhibiting the highest CALCRL expression compared to AML patients with the lowest CALCRL expression (data not show). To functionally investigate the role of CALCRL in LSC biology, we performed Limit Dilution Assay (LDA) from sorted CALCRL^{negative} and CALCRL^{positive} cell subpopulations (data not show) followed by injection of increasing cell doses in immunocompromised NSG mice and showed that the CALCRL^{positive} cell subpopulation was enriched in LSCs compared with the CALCRL^{negative} one (data not show). Then we performed ex vivo assays knocking down CALCRL in 4 primary AML samples followed by LDA and demonstrated that in all tested samples CALCRL invalidation significantly decreased the frequency of LSCs in vivo both in primary and secondary transplantations (data not show), demonstrating its requirement in preserving the function of LSCs.

Depletion of CALCRL Alters Cell Cycle and DNA Repair Pathways in AML

To examine regulatory pathways downstream of CALCRL, we generated and performed comparative transcriptomic and functional assays on shCTR vs shCALCRL MOLM-14 cells. CALCRL knockdown is associated with a significant decrease in the expression of 623 genes and an increase of 278 (FDR<0.05) (data not show). Data mining analyses showed significant depletion in genes involved in cell cycle and DNA integrity pathways in shCALCRL AML cells (data not show). Western blotting confirmed that CALCRL knockdown affected the expression of RAD51, CHEK1 and BCL2 protein levels in MOLM-14 and OCI-AML3 cells, in particular in the former (data not show). This was associated with an accumulation of cells in the G₀/G₁ phase (data not show). Interestingly, enrichment analysis showed that depletion of CALCRL affects the gene signatures of several key transcription factors such as E2F1, P53 or FOXM1 described as critical cell cycle regulators (data not show). We focused on the E2F1 transcription factor, whose importance in the biology of leukemic stem/progenitors cells has recently been shown (Pelicano et al., 2018). We first confirmed that CALCRL depletion was closely associated with a significant decrease in the activity of E2F1 (data not show). Then, we demonstrated that the knockdown of E2F1 affected protein expression of RAD51, CHK1 but not BCL2 (data not show), inhibited cell proliferation (data not show), cell cycle progression (data not show) and induced cell death in both MOLM-14 and OCI-AML3 (data not show). We further investigated whether CALCRL might regulate the proliferation of primary AML cells. Interestingly, CALCRL protein level positively correlated with clonogenic capacities in methylcellulose (data not show). As expected the depletion of CALCRL in primary samples decreased the number of colonies (data not show), and BCL2 and RAD51 protein levels (data not show). All these results suggested that CALCRL had a role in the proliferation of AML blasts and controls critical pathways involved in DNA repair processes.

CALCRL Downregulation Sensitizes Leukemic Cells to Chemotherapeutic Drugs Cytarabine and Idarubicin

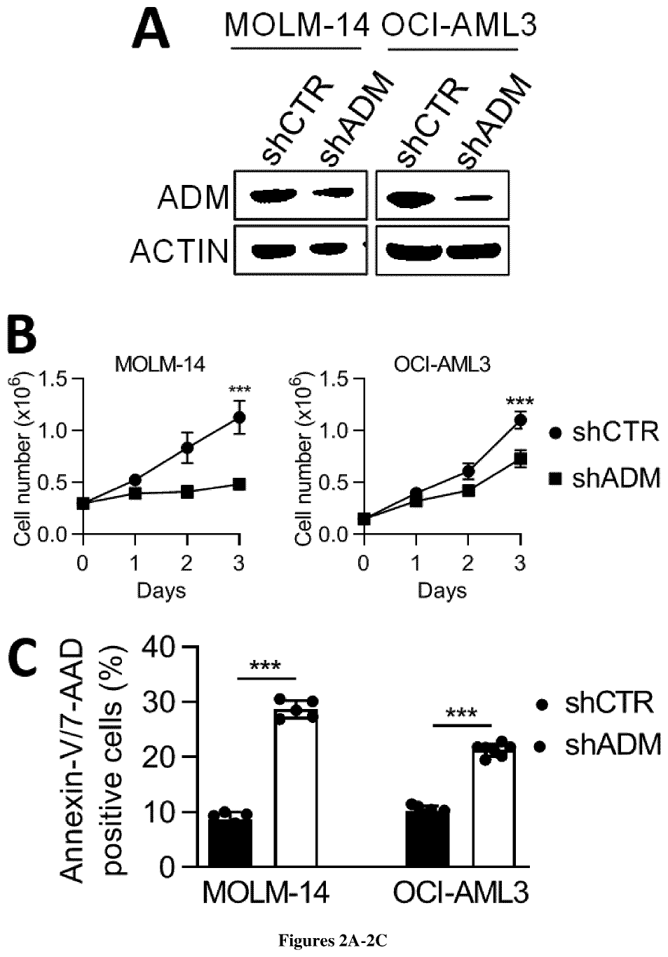

Based on CALCRL-regulated target proteins such as BCL2, CHK1 or FOXM1 (David et al., 2016; Khan et al., 2017; Konopleva et al., 2016), we hypothesized that CALCRL was involved in chemoresistance. Accordingly, CALCRL depletion sensitized MOLM-14 and OCI-AML3 cells to AraC and idarubicin through the reduction of cell viability (data not show) and the induction of cell death (increased AnnV staining, data not show; and increased cleavage of apoptotic proteins Caspase-3 and PARP, data not show). Furthermore, the depletion of ADM or E2F1 also sensitized AML cells to these genotoxic agents (FIG. 3). This demonstrated that the ADM-CALCRL-E2F1 axis plays a role in the chemoresistance in vitro. Importantly, siRNA-mediated depletion of CALCRL in seven primary AML samples combined with AraC significantly reduced clonogenic capacities of cells compared with siCTR+AraC and siCAL conditions. In order to confirm these results in vivo, we used our xenograft model of lentiviral inducible shRNA targeting CALCRL. After in vitro validation showing that this inducible shRNA recapitulated the chemosensitization observed with constitutive shRNAs (data not show), MOLM-14 cells transduced with shCTR or shCAL #2 were injected into immunodeficient NSG mice. After 10 days when the disease was well-established, shRNA-based depletion was induced and the mice were treated with 30 mg/kg/day AraC for 5 days (data not show). AraC in combination with shCALCRL significantly reduced the total number of blasts (data not show), induced a higher rate of cell death (data not show), and prolonged mice survival (data not show) compared to shCTR+AraC, shCTR alone or shCALCRL alone. Furthermore, MOLM-14 cells expressing shCTR and treated with vehicle or AraC were FACS-sorted and plated in vitro for further experiments. Interestingly, after one week of in vitro culture, human AML cells from AraC treated mice were more resistant to AraC (EC50: 2 μM for vehicle group vs 7 μM for AraC treated group) and idarubicin (EC50: 29 nM for vehicle group vs 61 nM for AraC treated group) (data not show). Next, we observed that AML cells treated with AraC in vivo had higher protein expression levels of CALCRL, and a slight increase in RAD51 and BCL2, whereas CHK1 was similar to untreated cells (data not show).

To evaluate the role of CALCRL in this chemoresistance pathway in vivo, we depleted CALCRL in these cells. Knockdown of CALCRL by two different shRNAs sensitized cells to AraC and idarubicin compared to shCTR in cells treated with vehicle (data not show) or AraC alone (data not show). Remarkably, the EC50 of AraC and idarucibin in AraC-treated cells in vivo and transduced with shCALCRL was similar to that observed for MOLM-14 cells from vehicle-treated mice and then transduced with shCTR. Because mitochondrial metabolism has emerged as a critical regulator of cell proliferation and survival in basal and chemotherapy-treated conditions in AML (Li et al 2019; Molina et al., 2018; Scotland et al., 2013; Sriskanthadevan et al., 2015; Farge et al., 2017; Laganidou et al 2013; Jones et al 2018, Pollyea et al 2019), we also analyzed the impact of CALCRL depletion on mitochondrial function. GSEA showed a significant depletion in the gene signature associated with mitochondrial oxidative metabolism in the shCALCRL MOLM-14 cells (data not show). OCR measurements revealed a modest but significant reduction in basal OCR, whereas maximal respiration was conserved, indicating that mitochondria remain functional (data not show). We also consistently observed a significant decrease in mitochondrial ATP production by shCALCRL (data not show). We and other groups have previously shown that chemoresistant cells have elevated oxidative metabolism and that targeting mitochondria in combination with conventional chemotherapy may be an innovative therapeutic approach in AML (Lagadinou et al 2013; Farge et al., 2017; Kuntz et al., 2017). Since the depletion of CALCRL modestly decreased OCR and more greatly decreased mitochondrial ATP in AML cells (data not show), we assessed cellular energetic status associated with AraC. Knockdown of CALCRL significantly abrogated the AraC-induced increase of basal respiration and maximal respiration (data not show). Moreover, we observed a decrease in mitochondrial ATP production in response to AraC upon CALCRL silencing (data not show) whereas glycolysis (e.g. ECAR) was not affected (data not show).

As it has been reported that BCL2 controlled oxidative status in AML cells (Lagadinou et al., 2013), we investigated its role downstream of CALCRL. We showed that upon AraC treatment, the overexpression of BCL2 in MOLM-14 cells (data not show) is sufficient to partially rescue maximal respiration but not basal respiration (data not show). This suggests a role of the CALCRL-BCL2 axis in maintaining some aspects of mitochondrial function in AraC resistant AML cells in response to AraC. This was not related to energy production, as neither mitochondrial ATP production nor ECAR were affected (data not show). Finally, BCL2 overexpression almost entirely inhibited basal apoptosis induced by the depletion of CALCRL and by the combination with AraC or idarubicin (data not show).

Overall, these results suggest that CALCRL and its downstream signaling pathways mediate chemoresistance of AML cells in a OXPHOS and BCL2-dependent manner.

Depletion of CALCRL in Residual Disease after AraC Treatment Impedes LSC Function

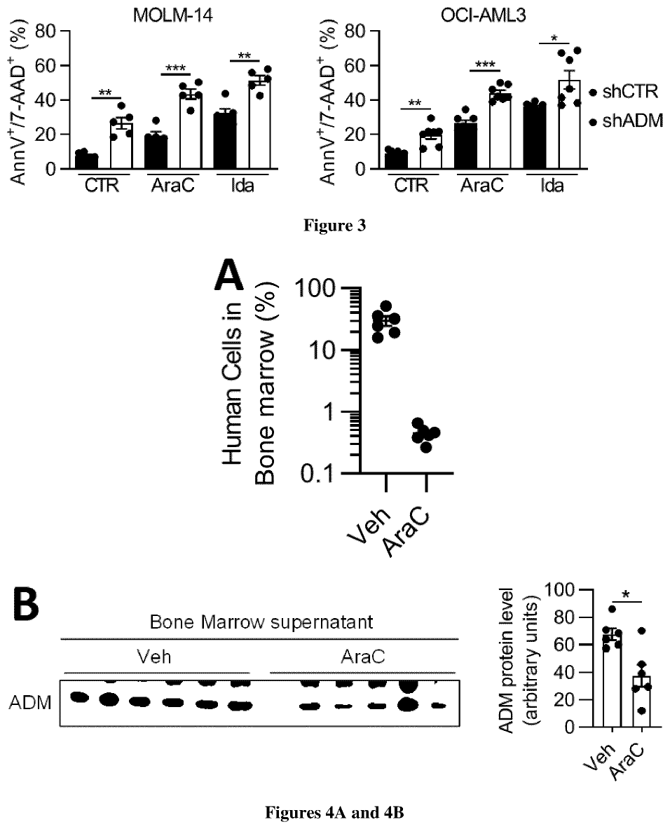

To address the role of CALCRL in response to chemotherapy in primary AML samples, we used a clinically relevant PDX model of AraC treatment in AML (Farge et al., 2017). After injection of primary cells into NSG mice and after engraftment was established and confirmed, mice were treated for 5 days with 60 mg/kg/day of AraC and sacrificed at day 8 to study the minimal residual disease (MRD; data not show). We tested and analyzed 10 different PDX models and stratified them according to their response to AraC as low (FC AraC-to-Vehicle<10) or high (FC>10) responders (data not show). The percentage of cells positive for CALCRL in AML bulk was approximately doubled in the low responder group compared to the high responder group (3.6% vs 7.8%; data not show C). We also observed an inverse linear correlation between the percentage of positive cells and the tumor reduction (R²=0.418; p=0.0434; data not show). Moreover, after AraC treatment a significant increase in the percentage of positive blasts for CALCRL was observed (5.6% vs. 23%; data not show) in all CD34/CD38 subpopulations (data not show) from minimal residual disease. In addition we also observed in PDX models treated 3 days with Idarubicin (data not show) a slight enrichment in CALCRL^{positive} cells (data not show), and an inverse correlation between the expression of CALCRL and the tumor reduction (data not shown). Although further studies will be required to confirm these results, they suggest that CALCRL expression might also predict the response to anthracyclines. We next investigated the effects of AraC on ADM secretion. For this, we evaluated the protein level of ADM in bone marrow supernatants of mice treated with PBS or AraC. Chemotherapy reduced both percentage of human cells (FIG. 4A) and levels of secreted ADM (FIG. 4B) in the bone marrow of mice. This correlation between the tumor mass and the secretion of ADM reinforced the hypothesis of an autocrine secretion of ADM by leukemic blasts. Then, to translate PDX observations from bench to clinic, we examined cell surface expression of CALCRL in patients before and after intensive chemotherapy (7+3) (data not shown). As expected, treatment decreased dramatically the percentage of blasts in the bone marrow (data not shown), accompanied with a significant enrichment in CALCRL^{positive} cells (data not shown). Moreover, in a single patient analysis, we observed a continuous increase in CALCRL^{positive} cells following chemotherapy (diag 12.9%, D35 32.8%, Rel 81%; data not shown). Recently Shlush et al. proposed an elegant model of relapses with two situations: in the first one called “relapse origin-primitive” (ROp), relapse originated from rare LSC clones only detectable in HSPC or after xenotransplantation. In the second model, called “relapse origin-committed” (ROc), relapse clone arose from immunophenotypically committed leukemia cells in which bulk cells harbored a stemness transcriptional profile (Shlush et al., 2016). We analyzed this transcriptomic database and observed that at the time of diagnosis, CALCRL expression was higher in blasts with ROc than with ROp phenotype, in accordance with the expression of CALCRL in cells harboring stem cell features (data not shown). Interestingly, CALCRL was strongly increased at relapse in ROp patients, which correlated with the emergence at this stage of the disease of a clone with stem cell properties data not shown). These observations supported our hypothesis of the preexistence of a relapse-relevant LSC population, rare (ROp) or abundant (ROc), expressing high levels of CALCRL. Finally, to definitively determine the role of CALCRL in the maintenance of R-LSCs, primary AML cells were injected into NSG mice, and after engraftment and treatment with AraC, human viable AML cells constituting the minimal residual disease was collected and then transfected with siCTR or siCALCRL before transplantation into secondary recipients with LDA to determine the frequency of LSCs (data not shown). A significant reduction of LSC frequency was observed in the siCALCRL treatment compared to the siCTR in the bone marrow of two primary AML samples (data not shown). Altogether, these results strongly supported the conclusion that CALCRL preserved LSC function after chemotherapy and that it was an attractive therapeutic target to eradicate the clone at the origin of relapse (data not shown).

### DISCUSSION

Clinical efficacy of LSC-selective targeted therapies has not been proven for AML treatment due to high plasticity and heterogeneity not only for the phenotype (Taussig et al., 2010; Eppert et al., 2011; Sarry et al., 2011) but also for the drug sensitivity (Farge et al., 2017; Boyd et al., 2018) of the LSC population. However, fundamental studies focusing on intrinsic properties of this cell population such as their resistance to chemotherapy are crucially needed for the development of improved and more specific therapies in AML.

Our study provides key insights of LSC biology and drug resistance and identifies the ADM receptor CALCRL as a master regulator of R-LSCs. Our work first shows that CALCRL gene is overexpressed in the leukemic compartment compared to normal counterpart based on Eppert's study that functionally characterizes LSCs. CALCRL could be specifically upregulated by LSC-related transcription factors such as HIF1α or ATF4 (Wang et al., 2011; van Galen et al., 2018). Indeed, both ADM and CALCRL possess the consensus hypoxia-response element (HRE) in the 5′-flanking region and are HIF1α-regulated genes (Nikitenko et al., 2003). Recently, it has been demonstrated that the integrated stress response and the transcription factor ATF4 is involved in AML cell proliferation and is uniquely active in HSCs and LSCs (van Galen et al., 2018; Heydt et al., 2018). Interestingly, maintenance of murine HSCs under proliferative stress but not steady-state conditions is dependent on CALCRL signaling (Suekane et al., 2019). Accordingly, CALCRL might support leukemic hematopoiesis and overcome stress induced by the high proliferation rate of AML cells.

Our findings clearly show that targeting CALCRL expression impacts clonogenic capacities, cell cycle progression and genes related to DNA repair and genomic stability. If cancer stem cells and LSCs are predominantly quiescent thereby preserving them from chemotherapy, recent studies suggested that LSCs also display a more active cycling phenotype (Iwasaki et al., 2015; Pei et al., 2018). C-type lectin CD93 is expressed on a subset of actively cycling, non-quiescent AML cells enriched for LSC activity (Iwasaki et al., 2015). Recently, Pei et al. showed that targeting the AMPK-FIS1 axis disrupted mitophagy and induced cell cycle arrest in AML, leading to the depletion of LSC potential in primary AML. These results are consistent with the existence of different sub-populations of LSCs that differ in proliferative state. Moreover, FIS1 depletion induces the down-regulation of several genes (e.g. CCND2, CDC25A, PLK1, CENPO, AURKB) and of the E2F1 gene signature that both we also identified after CALCRL knockdown. Recently, it has been proposed that E2F1 plays a pivotal role in regulating CML stem/progenitor cells proliferation and survival status (Pellicano et al., 2018).

Several signaling pathways, for instance MAPKs, CDK/cyclin or PI3K/AKT, have been described to be stimulated by ADM/CALCRL axis and may control pRB/E2F1 complex activity (Hallstrom et al., 2008; Wang et al., 1998). Other signaling mediators activated in LSCs such as c-Myc and CEBPα regulate E2F1 transcription and allow the interaction of the E2F1 protein with the E2F gene promoters to activate genes essential for DNA replication at G1/S, cell proliferation and survival in AML (Leung et al., 2008; O'Donnell et al., 2005; Rishi et al., 2014). Therefore, this analysis of cellular signaling downstream of CALCRL uncovers new pathways crucial for the maintenance and the chemoresistance of LSCs.

The characterization of chemotherapy-spared R-LSCs, which are present at the onset of relapse, is necessary to develop new therapeutic strategies to eradicate them. Boyd and colleagues have proposed the existence of a transient state of Leukemic Regenerating Cells (LRC) during the immediate and acute response to AraC that are responsible for disease regrowth foregoing the recovery of LSC pool (Boyd et al., 2018). In this attractive model and in the dynamic of MRD post-chemotherapy, CALCRL-positive AML cells are a part of this LRC subpopulation and CALCRL is essential for the preservation of LSC potential of chemoresistant primary AML. It would be interesting to determine whether chemotherapy only spares cells that are positive for CALCRL and/or whether it induces an adaptive response to stress that increases the expression of CALCRL. Transcription factors that are activated in response to chemotherapy should be identified to improve our knowledge of the acute response to chemotherapy. Therefore, therapeutic targeting of CALCRL should be clinically investigated to specifically eradicate MRD and prevent relapse in AML. Finally, as several molecules preventing the binding of the neuropeptide CGRP to CALCRL have been developed for treatment in other diseases (Hutchings et al., 2017; Schuster and Rapoport, 2017), this facilitates future pharmacological approaches to antagonize ADM-CALCRL axis in AML.

In summary, our data clearly identify CALCRL as a new stem cell actor required to sustain AML development in vivo. This receptor regulates genes involved in chemoresistance mechanisms and its depletion sensitizes AML cells to both cytarabine and anthracyclines in vitro and in vivo. This further indicates that LSCs resistant to these drugs share common activated pathways involved in these resistance mechanisms. All of these results strongly suggest CALCRL and AMD is a new and promising candidate therapeutic target for anti-LSC therapy.

### REFERENCES

Throughout this application, various references describe the state of the art to which this invention pertains. The disclosures of these references are hereby incorporated by reference into the present disclosure.
Berenguer-Daizé, C., Boudouresque, F., Bastide, C., Tounsi, A., Benyahia, Z., Acunzo, J., Dussault, N., Delfino, C., Baeza, N., Daniel, L., et al. (2013). Adrenomedullin blockade suppresses growth of human hormone-independent prostate tumor xenograft in mice. Clin. Cancer Res. 19, 6138-6150.
Bonnet, D., and Dick, J. E. (1997). Human acute myeloid leukemia is organized as a hierarchy that originates from a primitive hematopoietic cell. Nat. Med. 3, 730-737.
Bosc, C., Selak, M. A., and Sarry, J.-E. (2017). Resistance Is Futile: Targeting Mitochondrial Energetics and Metabolism to Overcome Drug Resistance in Cancer Treatment. Cell Metab. 26, 705-707.
Boyd, A. L., Aslostovar, L., Reid, J., Ye, W., Tanasijevic, B., Porras, D. P., Shapovalova, Z., Almakadi, M., Foley, R., Leber, B., et al. (2018). Identification of Chemotherapy-Induced Leukemic-Regenerating Cells Reveals a Transient Vulnerability of Human AML Recurrence. Cancer Cell 34, 483-498.e5.
Chen, Y., Jacamo, R., Konopleva, M., Garzon, R., Croce, C., and Andreeff, M. (2013). CXCR4 downregulation of let-7a drives chemoresistance in acute myeloid leukemia. J. Clin. Invest. 123, 2395-2407.
Daria, D., Kirsten, N., Muranyi, A., Mulaw, M., Ihme, S., Kechter, A., Hollnagel, M., Bullinger, L., Döhner, K., Döhner, H., et al. (2016). GPR56 contributes to the development of acute myeloid leukemia in mice. Leukemia 30, 1734-1741.
David, L., Fernandez-Vidal, A., Bertoli, S., Grgurevic, S., Lepage, B., Deshaies, D., Prade, N., Cartel, M., Larrue, C., Sarry, J.-E., et al. (2016). CHK1 as a therapeutic target to bypass chemoresistance in AML. Sci. Signal. 9, ra90-ra90.
Eppert, K., Takenaka, K., Lechman, E. R., Waldron, L., Nilsson, B., van Galen, P., Metzeler, K. H., Poeppl, A., Ling, V., Beyene, J., et al. (2011). Stem cell gene expression programs influence clinical outcome in human leukemia. Nat. Med. 17, 1086-1093.
Farge, T., Saland, E., de Toni, F., Aroua, N., Hosseini, M., Perry, R., Bosc, C., Sugita, M., Stuani, L., Fraisse, M., et al. (2017). Chemotherapy-Resistant Human Acute Myeloid Leukemia Cells Are Not Enriched for Leukemic Stem Cells but Require Oxidative Metabolism. Cancer Discov. 7, 716-735.
van Galen, P., Mbong, N., Kreso, A., Schoof, E. M., Wagenblast, E., Ng, S. W. K., Krivdova, G., Jin, L., Nakauchi, H., and Dick, J. E. (2018). Integrated Stress Response Activity Marks Stem Cells in Normal Hematopoiesis and Leukemia. Cell Rep. 25, 1109-1117.e5.
van Galen, P., Hovestadt, V., Wadsworth II, M. H., Hughes, T. K., Griffin, G. K., Battaglia, S., Verga, J. A., Stephansky, J., Pastika, T. J., Lombardi Story, J., et al. (2019). Single-Cell RNA-Seq Reveals AML Hierarchies Relevant to Disease Progression and Immunity. Cell 176, 1265-1281.e24.
Gentles, A. J. (2010). Association of a Leukemic Stem Cell Gene Expression Signature With Clinical Outcomes in Acute Myeloid Leukemia. JAMA 304, 2706.
Hackl, H., Steinleitner, K., Lind, K., Hofer, S., Tosic, N., Pavlovic, S., Suvajdzic, N., Sill, H., and Wieser, R. (2015). A gene expression profile associated with relapse of cytogenetically normal acute myeloid leukemia is enriched for leukemia stem cell genes. Leuk. Lymphoma 56, 1126-1128.
Hallstrom, T. C., Mori, S., and Nevins, J. R. (2008). An E2F1-dependent gene expression program that determines the balance between proliferation and cell death. Cancer Cell 13, 11-22.
Henkenius, K., Greene, B. H., Barckhausen, C., Hartmann, R., Marken, M., Kaiser, T., Rehberger, M., Metzelder, S. K., Parak, W. J., Neubauer, A., et al. (2017). Maintenance of cellular respiration indicates drug resistance in acute myeloid leukemia. Leuk. Res. 62, 56-63.
Heydt, Q., Larrue, C., Saland, E., Bertoli, S., Sarry, J.-E., Besson, A., Manenti, S., Joffre, C., and Mansat-De Mas, V. (2018). Oncogenic FLT3-ITD supports autophagy via ATF4 in acute myeloid leukemia. Oncogene 37, 787-797.
Ho, T.-C., LaMere, M., Stevens, B. M., Ashton, J. M., Myers, J. R., ODwyer, K. M., Liesveld, J. L., Mendler, J. H., Guzman, M., Morrissette, J. D., et al. (2016). Evolution of acute myelogenous leukemia stem cell properties after treatment and progression. Blood 128, 1671-1678.
Hutchings, C. J., Koglin, M., Olson, W. C., and Marshall, F. H. (2017). Opportunities for therapeutic antibodies directed at G-protein-coupled receptors. Nat. Rev. Drug Discov. 16, 787-810.
Ishikawa, F., Yoshida, S., Saito, Y., Hijikata, A., Kitamura, H., Tanaka, S., Nakamura, R., Tanaka, T., Tomiyama, H., Saito, N., et al. (2007). Chemotherapy-resistant human AML stem cells home to and engraft within the bone-marrow endosteal region. Nat. Biotechnol. 25, 1315-1321.
Iwasaki, M., Liedtke, M., Gentles, A. J., and Cleary, M. L. (2015a). CD93 Marks a Non-Quiescent Human Leukemia Stem Cell Population and Is Required for Development of MLL-Rearranged Acute Myeloid Leukemia. Cell Stem Cell 17, 412-421.
Iwasaki, M., Liedtke, M., Gentles, A. J., and Cleary, M. L. (2015b). CD93 Marks a Non-Quiescent Human Leukemia Stem Cell Population and Is Required for Development of MLL-Rearranged Acute Myeloid Leukemia. Cell Stem Cell 17, 412-421.
Jin, L., Lee, E. M., Ramshaw, H. S., Busfield, S. J., Peoppl, A. G., Wilkinson, L., Guthridge, M. A., Thomas, D., Barry, E. F., Boyd, A., et al. (2009). Monoclonal antibody-mediated targeting of CD123, IL-3 receptor alpha chain, eliminates human acute myeloid leukemic stem cells. Cell Stem Cell 5, 31-42.
Jordan, C. T., Guzman, M. L., and Noble, M. (2006). Cancer stem cells. N. Engl. J. Med. 355, 1253-1261.
Khan, I., Halasi, M., Zia, M. F., Gann, P., Gaitonde, S., Mahmud, N., and Gartel, A. L. (2017). Nuclear FOXM1 drives chemoresistance in AML. Leukemia 31, 251-255.
Kikushige, Y., Shima, T., Takayanagi, S., Urata, S., Miyamoto, T., Iwasaki, H., Takenaka, K., Teshima, T., Tanaka, T., Inagaki, Y., et al. (2010). TIM-3 is a promising target to selectively kill acute myeloid leukemia stem cells. Cell Stem Cell 7, 708-717.
Kocemba, K. A., van Andel, H., de Haan-Kramer, A., Mahtouk, K., Versteeg, R., Kersten, M. J., Spaargaren, M., and Pals, S. T. (2013). The hypoxia target adrenomedullin is aberrantly expressed in multiple myeloma and promotes angiogenesis. Leukemia 27, 1729-1737.
Konopleva, M., Pollyea, D. A., Potluri, J., Chyla, B., Hogdal, L., Busman, T., McKeegan, E., Salem, A. H., Zhu, M., Ricker, J. L., et al. (2016a). Efficacy and Biological Correlates of Response in a Phase II Study of Venetoclax Monotherapy in Patients with Acute Myelogenous Leukemia. Cancer Discov. 6, 1106-1117.
Konopleva, M., Pollyea, D. A., Potluri, J., Chyla, B., Hogdal, L., Busman, T., McKeegan, E., Salem, A. H., Zhu, M., Ricker, J. L., et al. (2016b). Efficacy and Biological Correlates of Response in a Phase II Study of Venetoclax Monotherapy in Patients with Acute Myelogenous Leukemia. Cancer Discov. 6, 1106-1117.
Kuntz, E. M., Baquero, P., Michie, A. M., Dunn, K., Tardito, S., Holyoake, T. L., Helgason, G. V., and Gottlieb, E. (2017). Targeting mitochondrial oxidative phosphorylation eradicates therapy-resistant chronic myeloid leukemia stem cells. Nat. Med. 23, 1234-1240.
Lagadinou, E. D., Sach, A., Callahan, K., Rossi, R. M., Neering, S. J., Minhajuddin, M., Ashton, J. M., Pei, S., Grose, V., O'Dwyer, K. M., et al. (2013). BCL-2 Inhibition Targets Oxidative Phosphorylation and Selectively Eradicates Quiescent Human Leukemia Stem Cells. Cell Stem Cell 12, 329-341.
Leung, J. Y., Ehmann, G. L., Giangrande, P. H., and Nevins, J. R. (2008). A role for Myc in facilitating transcription activation by E2F1. Oncogene 27, 4172-4179.
Li, L., Osdal, T., Ho, Y., Chun, S., McDonald, T., Agarwal, P., Lin, A., Chu, S., Qi, J., Li, L., et al. (2014). SIRT1 activation by a c-MYC oncogenic network promotes the maintenance and drug resistance of human FLT3-ITD acute myeloid leukemia stem cells. Cell Stem Cell 15, 431-446.
Majeti, R., Chao, M. P., Alizadeh, A. A., Pang, W. W., Jaiswal, S., Gibbs, K. D., van Rooijen, N., and Weissman, I. L. (2009). CD47 is an adverse prognostic factor and therapeutic antibody target on human acute myeloid leukemia stem cells. Cell 138, 286-299.
Molina, J. R., Sun, Y., Protopopova, M., Gera, S., Bandi, M., Bristow, C., McAfoos, T., Morlacchi, P., Ackroyd, J., Agip, A.-N. A., et al. (2018). An inhibitor of oxidative phosphorylation exploits cancer vulnerability. Nat. Med. 24, 1036-1046.
Ng, S. W. K., Mitchell, A., Kennedy, J. A., Chen, W. C., McLeod, J., Ibrahimova, N., Arruda, A., Popescu, A., Gupta, V., Schimmer, A. D., et al. (2016). A 17-gene stemness score for rapid determination of risk in acute leukaemia. Nature 540, 433-437.
Nikitenko, L. L., Smith, D. M., Bicknell, R., and Rees, M. C. P. (2003). Transcriptional regulation of the CRLR gene in human microvascular endothelial cells by hypoxia. FASEB J. 17, 1499-1501.
O'Donnell, K. A., Wentzel, E. A., Zeller, K. I., Dang, C. V, and Mendell, J. T. (2005). c-Myc-regulated microRNAs modulate E2F1 expression. Nature 435, 839-843.
Pabst, C., Bergeron, A., Lavallée, V.-P., Yeh, J., Gendron, P., Norddahl, G. L., Krosl, J., Boivin, I., Deneault, E., Simard, J., et al. (2016). GPR56 identifies primary human acute myeloid leukemia cells with high repopulating potential in vivo. Blood 127, 2018-2027.
Pei, S., Minhajuddin, M., Adane, B., Khan, N., Stevens, B. M., Mack, S. C., Lai, S., Rich, J. N., Inguva, A., Shannon, K. M., et al. (2018). AMPK/FIS1-Mediated Mitophagy Is Required for Self-Renewal of Human AML Stem Cells. Cell Stem Cell 23, 86-100.e6.
Pellicano, F., Park, L., Hopcroft, L. E. M., Shah, M. M., Jackson, L., Scott, M. T., Clarke, C. J., Sinclair, A., Abraham, S. A., Hair, A., et al. (2018). hsa-mirl83/EGR1-mediated regulation of E2F1 is required for CML stem/progenitor cell survival. Blood 131, 1532-1544.
Perna, F., Berman, S. H., Soni, R. K., Mansilla-Soto, J., Eyquem, J., Hamieh, M., Hendrickson, R. C., Brennan, C. W., and Sadelain, M. (2017). Integrating Proteomics and Transcriptomics for Systematic Combinatorial Chimeric Antigen Receptor Therapy of AML. Cancer Cell 32, 506-519.e5.
Quek, L., Otto, G. W., Garnett, C., Lhermitte, L., Karamitros, D., Stoilova, B., Lau, I.-J., Doondeea, J., Usukhbayar, B., Kennedy, A., et al. (2016). Genetically distinct leukemic stem cells in human CD34− acute myeloid leukemia are arrested at a hemopoietic precursor-like stage. J. Exp. Med. 213, 1513-1535.
Rishi, L., Hannon, M., Salome, M., Hasemann, M., Frank, A.-K., Campos, J., Timoney, J., O'Connor, C., Cahill, M. R., Porse, B., et al. (2014). Regulation of Trib2 by an E2F1-C/EBP feedback loop in AML cell proliferation. Blood 123, 2389-2400.
Saito, Y., Kitamura, H., Hijikata, A., Tomizawa-Murasawa, M., Tanaka, S., Takagi, S., Uchida, N., Suzuki, N., Sone, A., Najima, Y., et al. (2010). Identification of therapeutic targets for quiescent, chemotherapy-resistant human leukemia stem cells. Sci. Transl. Med. 2, 17ra9.
Sarry, J., Murphy, K., Perry, R., Sanchez, P. V, Secreto, A., Keefer, C., Swider, C. R., Strzelecki, A., Cavelier, C., Recher, C., et al. (2011). Human acute myelogenous leukemia stem cells are rare and heterogeneous when assayed in NOD/SCID/IL2R 7 c-deficient mice. 121.
Schuster, N. M., and Rapoport, A. M. (2017). Calcitonin Gene-Related Peptide-Targeted Therapies for Migraine and Cluster Headache. Clin. Neuropharmacol. 40, 169-174.
Scotland, S., Saland, E., Skuli, N., de Toni, F., Boutzen, H., Micklow, E., Sénégas, I., Peyraud, R., Peyriga, L., Theodoro, F., et al. (2013). Mitochondrial energetic and AKT status mediate metabolic effects and apoptosis of metformin in human leukemic cells. Leukemia 27, 2129-2138.
Shastri, A., Choudhary, G., Teixeira, M., Gordon-Mitchell, S., Ramachandra, N., Bernard, L., Bhattacharyya, S., Lopez, R., Pradhan, K., Giricz, O., et al. (2018). Antisense STAT3 inhibitor decreases viability of myelodysplastic and leukemic stem cells. J. Clin. Invest.
Sriskanthadevan, S., Jeyaraju, D. V, Chung, T. E., Prabha, S., Xu, W., Skrtic, M., Jhas, B., Hurren, R., Gronda, M., Wang, X., et al. (2015). AML cells have low spare reserve capacity in their respiratory chain that renders them susceptible to oxidative metabolic stress. Blood 125, 2120-2130.
Suekane, A., Saito, Y., Nakahata, S., Ichikawa, T., Ogoh, H., Tsujikawa, K., and Morishita, K. (2019). CGRP-CRLR/RAMP1 signal is important for stress-induced hematopoiesis. Sci. Rep. 9, 429.
Taussig, D. C., Vargaftig, J., Miraki-Moud, F., Griessinger, E., Sharrock, K., Luke, T., Lillington, D., Oakervee, H., Cavenagh, J., Agrawal, S. G., et al. (2010). Leukemia-initiating cells from some acute myeloid leukemia patients with mutated nucleophosmin reside in the CD34(−) fraction. Blood 115, 1976-1984.
Van Galen P., Hovestadt V, Wadsworth II M H, Hughes T K (2019). Single-Cell RNA-Seq Reveals AML Hierarchies Relevant to Disease Progression and Immunity. Cell 1265-1281.e24
Wang, S., Ghosh, R. N., and Chellappan, S. P. (1998). Raf-1 physically interacts with Rb and regulates its function: a link between mitogenic signaling and cell cycle regulation. Mol. Cell. Biol. 18, 7487-7498.
Wang, Y., Krivtsov, A. V, Sinha, A. U., North, T. E., Goessling, W., Feng, Z., Zon, L. I., and Armstrong, S. A. (2010). The Wnt/beta-catenin pathway is required for the development of leukemia stem cells in AML. Science 327, 1650-1653.
Wang, Y., Liu, Y., Malek, S. N., Zheng, P., and Liu, Y. (2011). Targeting HIFla eliminates cancer stem cells in hematological malignancies. Cell Stem Cell 8, 399-411.
Zeijlemaker, W., Grob, T., Meijer, R., Hanekamp, D., Kelder, A., Carbaat-Ham, J. C., Oussoren-Brockhoff, Y. J. M., Snel, A. N., Veldhuizen, D., Scholten, W. J., et al. (2018). CD34+CD38− leukemic stem cell frequency to predict outcome in acute myeloid leukemia. Leukemia.

1. A method of depleting leukemic stem cells in a subject suffering from acute myeloid leukemia (AML) comprising administering to the subject a therapeutically effective amount of an inhibitor of adrenomedullin activity or expression thereby depleting said leukemic stem cells.

2. A method for preventing relapse of a patient suffering from AML who was treated with chemotherapy comprising administering to the subject a therapeutically effective amount of an inhibitor of adrenomedullin activity or expression.

3. A method of treating chemoresistant acute myeloid leukemia (AML) in a patient in need thereof comprising administering to the patient a therapeutically effective amount of an inhibitor of adrenomedullin activity or expression.

4. The method of claim 3 wherein the leukemia is resistant to a combination of daunorubicin plus cytarabine (AraC), or idarubicin plus cytarabine (AraC).

5. The method according to claim 1, wherein the inhibitor of adrenomedullin activity is a polypeptide that binds to adrenomedullin and comprises all or a portion of the extracellular domains of CALCRL so as to form a soluble receptor that is capable of trapping adrenomedullin.

6. The method according to claim 1, wherein the inhibitor of adrenomedullin activity is an antibody that binds to adrenomedullin.

7. The method of claim 6 wherein the antibody binds to at least 2, 3, 4, 5, 6, 7, 8, 9, 10, or 11 consecutive amino acids located in the sequence which ranges from the amino acid residue at position 42 to the amino acid residue at position 52 in SEQ ID NO:1.

8. The method of claim 6 wherein the antibody binds to the sequence which ranges from the amino acid residue at position 42 to the amino acid residue at position 52 in SEQ ID NO:1.

9. The method according to claim 1, wherein the inhibitor of adrenomedullin expression is an antisense oligonucleotide or a siRNA that directly blocks the translation of the mRNA encoding for the precursor of adrenomedullin by binding thereto and thus preventing protein translation or increasing mRNA degradation, thus decreasing the level of adrenomedullin.
